(19) United States
(12) Patent Application Publication
(10) Pub. No.: US 2024/0382550 A1
(43) Pub. Date: Nov. 21, 2024

(54) AQUEOUS SOLUTION BASED ON SYZYGIUM AROMATICUM, AND ASSOCIATED PREPARATION METHOD AND USES

(71) Applicants: Charles DEBOUK, Cannes (FR); Eleia DBOUK, Cannes (FR)

(72) Inventor: Charles DEBOUK, Cannes (FR)

(21) Appl. No.: 18/696,023

(22) PCT Filed: Sep. 23, 2022
(86) PCT No.: PCT/EP2022/076507

§ 371 Date: Mar. 27, 2024

(30) Foreign Application Priority Data
Sep. 27, 2021 (FR) FR2110134

(51) Int. Cl. A61K 36/61; A61K 9/08; A61K 47/02

(52) U.S. Cl. CPC A61K 36/61; A61K 9/08; A61K 47/02; A61K 2236/333

(57) ABSTRACT

The subject matter of the invention is an aqueous solution for mucocutaneous administration, containing a plant matrix selected from plants or parts of plants of the Myrtaceae family in a solvent that is an aqueous solution with a pH of 7.5 or greater. The invention also covers a process for preparing such a solution. The solution according to the invention is used specifically in the prevention or treatment of infectious diseases.

### PRIOR ART

The present invention relates to an aqueous solution to be administered mucocutaneously. The invention also relates to a process for preparing this aqueous solution and its uses in the treatment or prevention of infectious diseases.

Millions of people suffer from respiratory diseases caused specifically by human pollution and also by various infectious diseases.

Infectious diseases are diseases caused by infectious agents. Infectious agents include viruses, bacteria, parasites and fungi.

The majority of these infectious agents succeed in infecting an organism through mucous membranes, such as the nasal, oral or ocular mucosa.

There is a wide spectrum of infectious diseases that can be transmitted from human to human or from animal to human.

To limit the transmission of these diseases, many preventive measures can be put in place to prevent these infections. This is the case, for example, with nasal washing, also known as nasal irrigation, which helps to prevent certain ENT (ear, nose and throat) infections such as sinusitis, rhinitis, common colds, flu and other respiratory tract infections.

Numerous clinical trials have successfully demonstrated that nasal washing is beneficial in combating these infections, especially in wintertime, when infectious diseases are most likely to spread.

Traditionally, nasal washing is performed using sprays or other medical devices that deliver an isotonic or physiological solution.

For example, patent application WO2018229556 discloses a composition for nasal and/or oral administration containing magnesium and one or more ingredients derived from a marine plant.

Many seawater-based products are already known, including Sterimar™, Marimer™ Humer™ salt solution, Respimer™, Physiomer™ or Prorhinel™.

However, use of these products leads to a level of discomfort, most notably unpleasant sensations of tingling or burning (caused specifically by a basic pH), which may bother the user. Also, daily use is required to achieve a satisfactory result.

Other products may also contain medicinal substances such as cortisone, which can be habit-forming and dangerous in cases of long-term use.

This has the effect of limiting the use of such products for the prevention and/or treatment of infectious diseases.

In addition, the prior art is known to contain various compositions designed to treat or prevent certain pathologies.

These compositions may comprise different ingredients of plant origin to treat or prevent the said diseases. For example, FR3032619 discloses a composition containing mallow to prevent and/or treat the symptoms of allergic rhinitis, while the article by Vicidomini et al., “Molecular Basis of the Therapeutical Potential of Clove and Clues to Its Anti-COVID-19 Utility” explores the therapeutic properties of clove against COVID-19.

The plant extracts mentioned in the literature are most often extracted using alcohol or a water-alcohol mixture as disclosed in application FR2641466, which describes a composition for prevention of medical treatment of periodontal disease specifically comprising a solvent consisting of water or a hydro-ethanol solution.

Similarly, document KR20200013005 describes on one hand a water-based drinkable food composition and on the other hand a composition to be inhaled for prevention or treatment of influenza and comprising medicinal herbs extracted using alcohol.

However, it is known that the alcohol in such compositions is generally used for the extraction of components such as essential oils. Such compositions may also be irritating if they are then applied directly to the skin and/or mucous membranes.

On the other hand, there also exist compositions comprising plant extracts obtained by mechanical processing such as extrusion. For example, document WO2016146838 describes a process for obtaining a plant matrix extract by mechanical treatment in an extruder in the presence of an aqueous solution. The extract thus obtained cannot be used as is, and is preferably used within a composition adapted to suite its method of administration.

### Technical Problem

In view of the above, one problem that the present invention proposes to solve is development of an effective alternative aqueous solution suitable for prevention or treatment of infectious diseases by limiting or avoiding any undesirable effects associated with its internal and external use.

This aqueous solution should preferably have analgesic, anti-inflammatory, anti-parasitic, anti-fungal and/or antibacterial properties.

Furthermore, this solution can be adapted and presented in the form of a composition suitable for topical use.

### Technical Solution

The solution to this problem is a solution administered mucocutaneously, characterised in that it comprises a plant matrix selected from plants or parts of plants of the Myrtaceae family in a solvent that is an aqueous solution with a pH of 7.5 or greater.

The invention also relates to a method of preparing this solution for mucocutaneous administration, consisting of:
a first step of bringing a plant matrix into contact with a solvent;
at least one second step, which is a phase of extracting the active components of interest from the plant matrix & solvent mixture; and
a step involving recovery of the solution thus obtained, which has a pH between 6 and 7.5,
characterised in that the plant matrix is selected from plants or parts of plants of the Myrtaceae family and in that the solvent is an aqueous solution with a pH of 7.5 or greater.

The invention also relates to a solution administered mucocutaneously and obtainable by the process according to the invention described above.

The invention also relates to a medical device containing a solution according to the invention.

A further object of the invention is a solution for use in the prevention or treatment of infectious diseases.

Another object is a topical composition in the form of a cream, gel or emulsion containing a solution according to the invention.

It is also designed to use a solution according to the invention for cleansing the ENT sphere.

### Advantages Obtained

The invention and the advantages arising therefrom will be better understood by reading the following description and the following non-limiting embodiments.

The Applicant has been able to demonstrate that the solution according to the invention specifically has one or more of the following advantages:
an anti-inflammatory effect;
an analgesic effect;
an anti-parasitic effect;
an anti-fungal effect;
an anti-bacterial effect;
a healing effect;
a preventive effect against infectious diseases; and/or
a curative effect on infectious diseases.

Furthermore, the solution developed by the Applicant is stable and well tolerated by the user and can also take the form of a composition suitable for topical use.

### DETAILED DESCRIPTION

In this description, unless otherwise specified, it is understood that when an interval is given, it includes the upper and lower limits of the said interval.

The subject matter of the invention is a solution to be administered mucocutaneously and containing a plant matrix selected from plants or parts of plants of the Myrtaceae family in a solvent that is an aqueous solution with a pH of 7.5 or greater.

The solution according to the invention contains a plant matrix selected from plants or parts of plants of the Myrtaceae family

By way of non-limiting examples, the plant matrix may be selected from plants or parts of plants of the genuses Amomyrtus, Angophora, Callistemon, Eucalyptus, Feijoa, Myrtus, Chamelaucium, Leptospermum, Metrosideros, Agonis, Astartea, Backhousia, Baeckea, Beaufortia, Calothamnus, Calyptranthes, Calytrix, Corymbia, Eugenia, Kunzea, Lophomyrtus, Luma, Melaleuca, Mosiera, Psidium, Syncarpia and Syzygium belonging to the Myrtaceae family.

Preferably, the plant matrix will be the species Syzygium aromaticum, also known as Eugenia caryophyllus.

Advantageously, the plant matrix is a plant or part of plant of the species Syzygium aromaticum. The plant parts that can be used are the cloves, stems, claws and leaves of Syzygium aromaticum.

Preferably, the plant matrix is a clove.

Syzygium aromaticum, also known as the clove tree, is a plant species found in Asia and South America.

The cloves that can be used as a plant matrix are the flower buds of the clove tree.

The species Syzygium aromaticum and its plant parts contain various molecules including terpenes and aromatic compounds. These molecules include eugenol, an aromatic compound known for its anti-inflammatory, analgesic, anti-parasitic, anti-fungal, antibacterial, anaesthetic and healing properties, as well as beta carophyllene, oleanic acid, alpha humulene, alpha copaene, furfural and vanillin. The pH of cloves is acidic and ranges from 2 to 3.5.

The solution according to the invention also contains a solvent which is an aqueous solution with a pH of 7.5 or greater.

The solvent is preferably chosen from sea water or any water to which salt may be added, such as fresh water, mineral water, spring water, demineralised water or thermal water. The salt that can be used is selected from sodium chloride or sodium bicarbonate.

Preferably, the solvent used should not be an alcohol. An alcohol is defined as any organic compound in which one of the carbon atoms is linked to a —OH hydroxyl group.

Preferably, the solvent used will be sea water.

By way of non-limiting examples, the seawater used can be water from the seas, gulfs and straits of the Atlantic Ocean, the Arctic Ocean, the Pacific Ocean, the Indian Ocean or the Southern Ocean.

Seawater is a rich source of various trace elements and minerals, including magnesium, bromine, chloride, sodium, sulphate, calcium, potassium, bromide, boron, strontium, fluoride, zinc, cadmium, iron, cobalt, copper, chromium and manganese in various concentrations. Seawater also contains gases dissolved in water, such as nitrogen, oxygen, argon or carbon dioxide.

In an alternative embodiment, the solvent used is fresh water with sodium chloride added.

In another alternative embodiment, the solvent used is fresh water with sodium bicarbonate added.

The solution according to the invention is suitable for mucocutaneous administration.

Mucocutaneous administration means any application of the solution according to the invention to the skin or mucous membranes. Absorption is through the skin, by an epidermal or dermal route and through mucous membranes through the eye, ear, nose, mouth, vagina or anus without risk of discomfort on application, especially for use in contact with human and animal cells, without toxicity, irritation, undue allergic response or similar reaction, and consistent with a reasonable benefit/risk ratio.

The invention also relates to a method of preparing such a solution for mucocutaneous administration.

The first step of the said process is a step of bringing a plant matrix into contact with a solvent.

The plant matrix used in the process according to the invention is selected from plants or parts of plants of the Myrtaceae family. It is brought into contact with a solvent which is an aqueous solution with a pH of 7.5 or greater, preferably chosen from sea water or any water to which salt may be added, such as fresh water, mineral water, spring water, demineralised water or thermal water. The salt, which may also be added at a later stage, is preferably chosen from sodium chloride or sodium bicarbonate.

The temperature of the solvent used in the first process step is preferably between 5° C. and 100° C. This temperature can, for example, be reached using a refrigerator, a cooling cell, a hot plate, a kettle, a microwave oven or any other known means of cooling or heating.

Preferably, the temperature of the solvent is between 60° C. and about 100° C. Even more preferably, the temperature of the solvent is between 90° C. and 100° C. The particularly preferred values used by the Applicant are 90° C., 95° C. and 100° C.

The process contains at least a second step which is a phase of extraction of active compounds of interest from the plant matrix & solvent mixture produced during the first step.

The term “active compound of interest” refers to any biologically active molecule extracted in the second step. Preferably, the active compounds of interest are extracted from the plant Syzygium aromaticum.

Extraction involves separating elements (molecules) from a liquid (liquid/liquid extraction) matrix or solid (solid/liquid extraction) matrix, usually using a solvent.

Preferably, extraction is not carried out using a mechanical extraction process such as extrusion.

According to the invention, extraction can preferably be carried out by infusion, decoction or maceration.

Infusion involves bringing a solvent to boiling point or close to boiling point—usually that of water—and then pouring this solvent onto a plant matrix. Alternatively, the plant matrix can also be placed in a solvent that is at or near boiling point. Preferably, the infusion is covered to keep all the volatile substances in the infusion and prevent them from evaporating. After the solvent is boiled, the plant matrix is advantageously boiled for a defined period of time between one minute and ten minutes, preferably two minutes. The mixture is then infused without a heat source for a specified period of time, which can vary from 1 to about 24 hours, preferably between 2 and 12 hours; the infusion is filtered and a brew is recovered.

Decoction involves soaking the plant matrix in a cold or ambient temperature solvent—usually water—and bringing the mixture to or close to boiling point, boiling for between 1 minute and 2 hours, and preferably about ten minutes. The mixture is then filtered to obtain a decoction.

Maceration in water involves soaking the plant matrix in cold water for several hours, preferably between 2 and 72 hours, and more preferably for between 4 and 24 hours. This method is suitable for plants in which compounds are labile and cannot tolerate high temperatures.

Extraction can also be achieved by using ultrasound or microwaves.

Ultrasonic assisted extraction is an extraction process that can be performed from a solid matrix to a liquid phase or between two liquid phases. The ultrasound waves used are over 20 KHz. This method of extraction allows thermolabile compounds to be preserved.

Microwave-assisted extraction is a solid-liquid extraction that can be carried out using a microwave oven and allows compounds to be extracted more quickly using heat. This extraction method has a lower solvent consumption but does not preserve certain thermolabile compounds.

Preferably, the second step of the process according to the invention is an extraction phase performed using infusion.

The active compounds of interest in the plant matrix & solvent mixture are extracted in the second step during the infusion time, which is preferably between 1 and 24 hours, preferably between 2 and 12 hours, and more preferably 7, 8, 9, 10 or 11 hours.

Finally, the process according to the invention contains a step of recovery of the solution thus obtained, with a pH between 6 and 7.5.

After this step of recovering the solution according to the invention, the solution is shaped in homogeneously in order to develop a product that can be marketed.

Advantageously, the process according to the invention may contain an additional step of filtering the solution obtained in the second step, prior to the step of recovering the solution thus obtained.

This filtration step involves recovering the plant matrix, preferably the cloves, that have been infused.

According to the invention, the plant matrix is selected from plants or parts of plants of the Myrtaceae family and the solvent is an aqueous solution with a pH of 7.5 or greater.

Preferably, the plant matrix is a plant or part of plant of the species Syzygium aromaticum, preferably clove, obtained from the flower buds of the clove tree, and the solvent is a saline solution, preferably sea water with a pH of between 8 and 8.5 and a salinity of between 3 and 300 g/1.

The term “salinity” refers to the amount of salt in the solvent. According to the invention, the salt can be sodium chloride or sodium bicarbonate.

The salinity of the solvent is advantageously adapted in order to obtain a neutral solution according to the invention with a pH of between 6 and 7.5 and preferably a neutral pH of 7.

Preferably, the salinity of the solvent is between 3 and 200 g/1. Even more preferably, the salinity of the solvent is between 3 and 150 g/1. The particularly preferred values used by the Applicant are 5 g/1, 10 g/1, 20 g/1, 35 g/1, 50 g/l and 100 g/1.

Similarly, the amount of plant matrix used, preferably cloves, can be adapted in order to obtain a neutral solution according to the invention with a pH of between 6 and 7.5 and preferably a neutral pH of 7.

Preferably, the concentration of plant matrix in the solution is between 0.1% and 15% by weight of the total weight of the solution. More preferably, the concentration is between 0.2% and 7.5% by weight of the total weight of the solution. Even more preferably, the concentration is between 0.5% and 5%. Much more preferably, the concentration is between 1.5% and 3%. The concentrations particularly preferred by the Applicant are 1.8%, 2%, 2.3%, 2.5%, 2.7% and 2.8%, preferably 2.3%, by weight of the total weight of the solution.

The pH of the solution can be monitored using a pH meter, pH paper strips or any other device used for measuring pH.

In a preferred embodiment, the solution obtained according to the invention contains an aqueous extract of the selected plant matrix and does not contain an oily extract. More preferably, the solution obtained according to the invention does not contain essential oil of cloves. The solution according to the invention suitable for mucocutaneous administration can be obtained using the process according to the invention.

In a particular embodiment of the solution according to the invention, the solution may contain at least one fragrant compound.

The term “fragrant compound” means a natural plant extract obtained from a plant; it also means a synthetic fragrant compound, that is, a compound chemically identical to a natural fragrant compound and obtained by synthesis, taken alone or in a mixture with other natural or synthetic fragrant compounds.

Preferably, the fragrant compound can be selected from coconut, argan, almond, amber, mint, eucalyptus and vanilla.

The solution according to the invention is advantageously introduced into a medical device.

According to the invention, the term “medical device” means any instrument, apparatus, equipment or other item, designed by the manufacturer to be used specifically for cosmetic and/or therapeutic purposes in humans for the prevention, control, treatment or alleviation of disease.

In one embodiment of the invention, the medical device used takes the form of drops or a spray suitable for dispensing the solution according to the invention via the ears, nose or mouth.

Surprisingly, the Applicant was also able to demonstrate that the solution according to the invention can be adapted for topical administration.

The invention therefore also relates to a topical composition in the form of a cream, gel or emulsion containing the solution according to the invention.

The term “topically administrable composition” refers to the act of applying or spreading the composition containing the solution according to the invention on the surface of the skin, a mucous membrane or dander.

“Skin” refers to the skin of the face and also of the whole body.

The composition according to the invention is formulated in a form suitable for topical application, containing a cosmetically or dermatologically acceptable medium, that is, one that has a pleasant colour, scent and feel and does not generate unacceptable levels of discomfort (tingling, tightness, redness), likely to deter consumers from using this composition.

The term “dander” refers to naturally occurring substances in a human or animal body that are rich in keratin, particularly hair, eyelashes, eyebrows and nails.

In an alternative embodiment, the composition according to the invention may also take the form of a lotion, foam, ointment, salve or poultice.

The solution or device according to the invention can be used to prevent or treat infectious diseases.

The term “infectious disease” refers to all diseases caused by infectious agents.

Non-limiting examples of infectious diseases include coronavirus infections such as COVID-19 or adenovirus, colds, flu, sore throat, bronchitis, sinusitis, rhinitis, tuberculosis, hepatitis, measles, malaria, borreliosis, fungal disease, gastroenteritis or toxoplasmosis.

According to an alternative method of use of the invention, it can also be used to prevent or treat infectious animal diseases caused by parasites, in particular nasal infections caused by Oestrus ovis in sheep (oestrosis) or the infection caused by Pneumonyssoides caninum in canines.

The term “parasite” refers to any animal or plant organism that can only live at the expense of a host organism of a different species, either permanently or during a phase of its life cycle.

In a preferred embodiment of the invention, the solution can be used for cleansing the ENT area.

The ENT sphere (short for ear, nose and throat) includes the throat, mouth, tongue, larynx, trachea, ears, sinuses and nose.

Regular nasal cleansing removes infectious agents and mucus that can block the nasal passages, making breathing difficult.

Nasal cleansing also helps to prevent dryness and drain and thin nasal secretions.

In a particular embodiment of the invention, the solution according to the invention can be used locally by diffusion into the nostrils in order to perform nasal washing.

The solution according to the invention, preferably combining seawater with a basic pH and cloves with an acid pH, is a solution with a neutral pH which, when used as a nasal cleansing solution, helps moisten and cleanse the nasal cavities by helping to maintain or restore the physiological balance of the mucous membranes and by providing minerals and trace elements. The solution according to the invention acts as a decongestant and/or antiseptic and helps remove volatile or allergenic pollutants such as pollen, dust, mites or fine particles.

Preferably, the solution according to the invention has a preventive and curative effect on infectious diseases.

The solution according to the invention also has the advantage of being less restrictive in terms of application, allowing longer intervals between washes than a conventional saline solution, and not being habit-forming or producing side effects when used regularly.

The solution according to the invention can also be used to cleanse the mouth and larynx, especially as a gargle.

According to another alternative method of use of the invention, it is suitable for internal use, in particular to relieve dental infections such as caries, mouth ulcers, gingivitis or periodontitis.

According to another alternative method of use of the invention, it may be adapted for use in preventing or treating dermatological diseases such as fungal skin infections or scalp peeling.

In particular, the solution according to the invention helps soothe the scalp and reduce dandruff.

Other examples of dermatological conditions include:
dermatological conditions with an inflammatory immunoallergic component, with or without a cell proliferation disorder, and in particular all forms of psoriasis, whether affecting the skin, the mucous membranes or the nails, and even psoriatic arthritis or atopic dermatitis and various forms of eczema;
healing disorders, or to promote healing in cases of wounds or burns.

According to another alternative method of use of the invention, it can be adapted for use in preventing or treating inflammatory diseases such as rheumatism, muscle pain or haemorrhoids. More particularly, the composition containing the solution according to the invention specifically provides an anti-inflammatory effect, a healing effect and/or a preventive effect on haemorrhoids.

Preferably, the topical composition comprising the solution according to the invention is adapted to prevent or treat infectious, dermatological and/or inflammatory diseases.

### EXAMPLES

This invention will now be illustrated with the following examples.

### Example 1: Process for Manufacture of a Solution a According to the Invention

The Applicant has developed the process for preparing the solution according to the invention.

23 g of cloves were used to make approximately two litres of the solution according to the invention.

The cloves are rinsed with clean water before use.

One litre of seawater (solvent) with a salinity of 35 g/l and a pH of 8 is placed in a covered container.

The container is placed on a heat source to heat the solvent to boiling point.

When the solvent boils, the cloves are added to the container and boiled for two minutes.

After two minutes of boiling, the heat source is turned off and the cloves are left to infuse overnight for a total of 8 hours.

After infusion, the cloves are filtered and recovered.

Advantageously, one litre of seawater solution enriched with natural active ingredients from cloves is obtained. To this, one litre of purified water is added in order to obtain two litres of the solution according to the invention.

Preferably, the solution is then left to stand for at least one hour to stabilise the pH.

Advantageously, the pH of the solution can be adjusted according to the invention thus obtained with the basic solvent (sea water or water with added salt (sodium chloride) or sodium bicarbonate) or by adding the plant matrix at acidic pH (plant matrix in solution or not) to obtain a neutral pH of 7.

Alternatively, the pH of the solution can also be altered by adding purified water.

The resulting solution A has a neutral pH of 7, a balance between the acidity of the clove and the alkalinity of the sea water.

The resulting solution A is then packaged in a container, such as a spray or dropper bottle, so that it can be administered to a user.

### Example 2: Efficiency Test on the Solution According to the Invention

A trial was carried out on 24 people, 12 of whom had a cold.

It is specified that all of these 24 people have no medical history likely to prevent them from taking part in this trial.

The 24 participants in this trial were randomly given a nasal spray containing either fresh water (control), a conventional saline solution, or a solution A according to the invention prepared according to the process described in Example 1.

The 24 people were thus classified into 6 groups:
Group 1: corresponds to the 4 people given a spray containing fresh water (control) and in good health;
Group 2: corresponds to the 4 people given a spray containing fresh water (control) and ill;
Group 3: corresponds to the 4 people given a spray containing a classic saline solution and in good health;
Group 4: corresponds to the 4 people given a spray containing a classic saline solution and ill;
Group 5: corresponds to the 4 people given a spray containing solution A according to the invention and in good health;
Group 6: corresponds to the 4 people given a spray containing solution A according to the invention and ill.

Each trial participant was asked to use the nasal spray given to them three times a week, preferably in the evening, to wash out their nose.

The period of use was set at 15 days.

After 15 days of use by all participants, each of them was asked the following question:
1: Is the product pleasant to use?
2A: Did you fall ill during the period in which the trial was carried out? (Question asked of healthy groups: Groups 1, 3 and 5).
2B: Did you notice an improvement in your health during the period when the trial was conducted? (Question asked of the sick groups: Groups 2, 4 and 6).
Group 1: 100% of the people given a fresh water solution answered yes to question 1 and 50% answered no to question 2A.
Group 2: 100% of the people given a fresh water solution answered yes to question 1 and 75% answered no to question 2B.
Group 3: 25% of the people given a conventional saline solution answered yes to question 1 and 50% answered no to question 2A.
Group 4: 25% of the people given a conventional saline answered yes to question 1 and 50% answered no to question 2B.
Group 5: 100% of the people given solution A according to the invention answered yes to question 1 and 75% answered no to question 2A.
Group 6: 100% of the people given solution A according to the invention answered yes to question 1 and 25% answered no to question 2B.

The Applicant has thus been able to demonstrate that the solution according to the invention, administered in the form of a nasal wash spray, is more pleasant to use than a conventional saline solution and shows a preventive effect on infectious diseases.

Similarly, the Applicant has been able to demonstrate that the solution according to the invention also has a curative effect on infectious diseases.

These results also show that the solution according to the invention has the advantage of being less restrictive in terms of application and allows the washings to be spaced further apart compared to a conventional saline solution.

### Example 3: Efficacy Test on the Composition Containing the Solution According to the Invention

The Applicant has been able to demonstrate that the solution according to the invention was also particularly well suited for topical administration. He has also developed a composition for this form of administration.

The format of the composition containing the solution according to the invention takes the form of a cream.

The topical composition was tested on a panel of eight people presenting symptoms of haemorrhoids. These symptoms include itching, burning and irritation, bleeding and haemorrhoidal episodes.

It is specified that all of these 8 people have no medical history likely to prevent them from taking part in this trial.

Each person was asked to use the composition containing the solution according to the invention two to three times a day for one week.

After one week of use of the composition containing the solution according to the invention, all eight people experienced a clear reduction in, or disappearance of, one or more haemorrhoid-related symptoms.

75% of the participants reported an improvement in their condition.

The main effects are soothing of itching, soothing of burning sensations, help with scar formation, cessation of bleeding and soothing during haemorrhoidal episodes.

1. Method of preparing a solution for mucocutaneous administration consisting of:
mixing a plant matrix comprising a plant or a part of plant of the species Syzygium aromaticum with a saline solution having a pH of between 8 and 8.5 and a salinity of between 3 g/l and 300 g/l to form a mixture;
extracting an active component from the mixture into a solution having a pH between 6 and 7.5; and
recoverin the solution thus obtained.

2. The method of claim 1, wherein the plant matrix is clove obtained from the flower buds of the clove tree, and the saline solution is sea water with a pH of between 8 and 8.5 and a salinity of between 3 g/l and 300 g/l.

3. The method of claim 1, comprising filtering the solution prior to recovering the solution.

4. The solution for mucocutaneous administration of claim 5, wherein the solution is obtained through the method of claim 1.

5. A solution for mucocutaneous administration comprising a plant matrix selected from plants or parts of plants of the species Syzygium aromaticum in a solvent that is a saline solution with a pH of 7.5 or greater.

6. A medical device containing a solution for mucocutaneous administration comprising a plant matrix selected from plants or parts of plants of the species Syzygium aromaticum in a solvent that is a saline solution with a pH of 7.5 or greater.

7. The medical device of claim 6, wherein the device takes the form of a nose, ear or mouth dropper or spray bottle.

8. The solution of claim 5, wherein the solution is a treatment of urinary incontinence.

9. The solution of claim 5, wherein the solution is in the form of a topical composition.

10. The solution of claim 9, wherein the solution is a treatment of urinary incontinence.

11. The solution of claim 9, wherein the topical composition is in the form of a cream, gel, or emulsion.
